(19) United States
(12) Patent Application Publication
(10) Pub. No.: US 2008/0255627 A1
(43) Pub. Date: Oct. 16, 2008

(54) Implantable Heart Stimulation Device and Method

(76) Inventor: Anders Bjorling, Solna (SE)

Correspondence Address: SCHIFF HARDIN, LLP;PATENT DEPARTMENT, 6600 SEARS TOWER, CHICAGO, IL, 60606-6473, US

(21) Appl. No.: 12/093,890

(22) PCT Filed: Nov. 23, 2005
(86) PCT No.: PCT/SE2005/001762

§ 371 Date: May 15, 2008

(51) Int. Cl. A61N 1/368

(52) U.S. Cl. 607/11; 607/14

(57) ABSTRACT

The above object is achieved in accordance with the present invention by a cardiac stimulation device for stimulating a heart having a first ventricle and a second ventricle. The device includes a first ventricle sensing circuit that is configured to communicate with a first ventricle sensing electrode suited to be positioned in or at the first ventricle, to enable the first ventricle sensing circuit to sense the first ventricle. The device also includes a second ventricle pacing circuit, configured to communicate with a second ventricle pacing electrode suited to be positioned in or at the second ventricle of the heart, to enable the second ventricle pacing circuit to pace the second ventricle. The device includes a control circuit that operates with time cycles corresponding to normal heart cycles. The control circuit is configured to, within such a time cycle, to detect a cardiac event in the first ventricle with the first ventricle sensing circuit and, after a time duration that is greater than or equal to zero, to cause the second ventricle pacing circuit to deliver a pacing pulse. The control circuit is also configured to detect the aforementioned loop of events indicative of the pacemaker-mediated tachycardia by detecting one or both of (i) the regularity of one or more repetitious events related to operation of the first ventricle sensing circuit or the second ventricle pacing circuit, and (ii) a repetitive operation pattern of the heart stimulation device. The control circuit is configured to determine if the detected regularity satisfies a predetermined regularity criterion and to determine if the operation pattern satisfies a predetermined operation pattern criterion. Based on whether one or both of these criteria are satisfied, the control circuit determines whether the aforementioned loop of events, and thus the presence of pacemaker-mediated tachycardia, is likely to exist.

### BACKGROUND OF THE INVENTION

1. Field of the Invention

The present invention relates to an implantable heart stimulation device with which it is possible to stimulate both the ventricles of a heart, i.e. a bi-ventricular pacer. The heart stimulation device can be a so-called pacemaker or an ICD (implantable cardioverter defibrillator) which also includes a pacing function.

The invention also relates to a method of, in a patient who is treated with a bi-ventricular cardiac heart stimulation device, detecting a pacemaker-mediated tachycardia of a certain kind.

2. Description of the Prior Art

Several different implantable devices for stimulating a heart are known. The devices are normally able to sense the electrical activity of the heart. Some implantable devices are able to deliver stimulation pulses to and/or sense the right atrium (in some cases even the left atrium) and also to deliver stimulation pulses to and sense both the left and right ventricles.

Devices that are able to deliver stimulation pulses to both the left and right ventricles can be called bi-ventricular pacers. Such devices can be used to treat patients who suffer from different severe cardiac problems, e.g. patients suffering from congestive heart failure (CHF). CHF is defined generally as the inability of the heart to deliver a sufficient amount of blood to the body. CHF can have different causes. It can for example be caused by a left bundle branch block (LBBB) or a right bundle branch block (RBBB). By using bi-ventricular pacing, the contraction of the ventricles can be controlled in order to improve the ability of the heart to pump blood. The stimulation pulses to the two ventricles can be delivered simultaneously but it is also known that the stimulation pulses to the two ventricles are delivered with a short time delay between them in order to optimize the pumping performance of the heart.

U.S. Pat. No. 5,720,768 describes different possible electrode positions in order to stimulate or sense the different chambers of the heart.

U.S. Pat. No. 6,070,100 describes that electrodes may be positioned in both the left and the right atrium as well as in the left and the right ventricles.

A problem that occurs in connection with heart stimulation devices is pacemaker-mediated tachycardia (PMT). A PMT is a phenomenon that is known in connection with a pacemaker that has both an atrial channel and a ventricular channel. A PMT of this kind is a reentry arrhythmia in which the pulse generator acts as the anterograde limb of the tachycardia and the natural conduction path acts as the retrograde limb. An R-wave (QRS-complex) is thus conducted to the atrium, causing a retrograde P-wave which is sensed by the device. The device then emits a ventricular pacing pulse after a certain delay (PV-delay). This pacing pulse captures the ventricle and the evoked R-wave is then conducted back to the atrium, and the cycle repeats itself. Such an endless loop between an atrium and a ventricle can of course also occur in a heart stimulation device that has two ventricular channels in addition to an atrial channel. U.S. Pat. No. 6,611,714 discloses a heart stimulation device that deals with such endless loop problems.

The articles “Pacemaker-Mediated Tachycardia in a Biventricular Pacing System” by van Gelder et al., PACE, vol. 24, December 2001, pp. 1819-1820 and “Cross-Ventricular Endless Loop Tachycardia During Biventricular Pacing” by Barold et al., PACE, vol. 24, December 2001, pp. 1821-1823 both describe that an endless loop tachycardia can occur if a dual chamber pacemaker (intended for sensing/pacing in an atrium and in a ventricle) is used as a bi-ventricular device.

### SUMMARY OF THE INVENTION

The invention is based on the recognition that a certain kind of pacemaker-mediated tachycardia can be a problem in a bi-ventricular cardiac heart stimulation device. This pacemaker-mediated tachycardia can be caused by the fact that a sensed event in a first ventricle causes the generation of a pacing pulse to the second ventricle, which pacing pulse causes a depolarization of the second ventricle with an associated electrical activity, which may also involve a repolarization, which electrical activity is transferred via the heart tissue to the first ventricle, and, after a certain time, is sensed in the first ventricle, which results in a new pacing pulse being applied to said second ventricle, which pacing pulse causes a depolarization of the second ventricle with an associated electrical activity, which may also involve a repolarization, which electrical activity is transferred via the heart tissue to the first ventricle, and, after a certain time, is sensed in the first ventricle, and so on.

An object of the present invention is to provide a cardiac heart stimulation device with which it is possible to detect a certain loop of events that if the heart stimulation device were (or is) in operation in a patient could be (or can be) a pacemaker-mediated tachycardia of the above kind.

The above object is achieved in accordance with the present invention by a cardiac stimulation device for stimulating a heart having a first ventricle and a second ventricle. The device includes a first ventricle sensing circuit that is configured to communicate with a first ventricle sensing electrode suited to be positioned in or at the first ventricle, to enable the first ventricle sensing circuit to sense the first ventricle. The device also includes a second ventricle pacing circuit, configured to communicate with a second ventricle pacing electrode suited to be positioned in or at the second ventricle of the heart, to enable the second ventricle pacing circuit to pace the second ventricle. The device includes a control circuit that operates with time cycles corresponding to normal heart cycles. The control circuit is configured to, within such a time cycle, to detect a cardiac event in the first ventricle with the first ventricle sensing circuit and, after a time duration that is greater than or equal to zero, to cause the second ventricle pacing circuit to deliver a pacing pulse. The control circuit is also configured to detect the aforementioned loop of events indicative of the pacemaker-mediated tachycardia by detecting one or both of (i) the regularity of one or more repetitious events related to operation of the first ventricle sensing circuit or the second ventricle pacing circuit, and (ii) a repetitive operation pattern of the heart stimulation device. The control circuit is configured to determine if the detected regularity satisfies a predetermined regularity criterion and to determine if the operation pattern satisfies a predetermined operation pattern criterion. Based on whether one or both of these criteria are satisfied, the control circuit determines whether the aforementioned loop of events, and thus the presence of pacemaker-mediated tachycardia, is likely to exist.

By configuring the control circuit to perform such detection and determination, it is possible to detect whether a PMT of the specific kind defined above is likely to be the case.

Preferably, the heart stimulation device according to the invention is an implantable heart stimulation device, i.e. a device that can be implanted in a human or animal being.

It should be noted that when it is stated herein that for example a certain circuit is adapted or configured to enable sensing and pacing of an atrium or ventricle, this does not necessarily mean that the circuit actually is connected to an atrium or a ventricle. Instead it means that if the heart stimulation device, in which the circuit in question is included, is actually implanted in a body with suitably located electrodes, then the circuit in question would be able to sense and pace an atrium or a ventricle. Similarly, the expressions relating to atrial or ventricular pacing and sensing circuits or the like only mean that these circuits are adapted to be able to sense typical atrial or ventricular events and that they are able to deliver pulses which are of the kind that is typical for stimulating atria or ventricles. A “pacing pulse” or the like is thus a pulse with an energy and morphology which would make it suitable to pace the relevant heart chamber.

It should also be noted that the mentioned “first ventricle” is not necessarily the ventricle that is paced/sensed first, if there is a normal time gap VV between the sense/pace in one ventricle and the pace (which may also be inhibited), during the same time cycle, in the other ventricle. The expressions “first ventricle” and “second ventricle” or the like are thus primarily used in order to distinguish between the two ventricles (or between the two ventricular channels of the heart stimulation device). This fact will be further explained below, when the label VVᵢ is explained.

According to one embodiment of the heart stimulation device according to the invention, the control circuit is arranged such that the detection of the regularity involves detecting the regularity of one or more of the following repetitious events:

a) the time between consecutive sensed events of the first ventricle sensing circuit,
b) the time between consecutive pacing pulses delivered with the second ventricle pacing circuit,
c) the time between a pacing pulse delivered with the second ventricle pacing circuit and the subsequent sensed event of the first ventricle sensing circuit.

Such repetition events can be used in order to determine whether the mentioned PMT is likely to be the case.

According to a further embodiment of the heart stimulation device, the control circuit is configured such that the regularity criterion is that a measure of how much the respective time varies is below a predetermined value. If the respective time varies very little between different cycles, then it is likely that the mentioned PMT is the case.

According to a further embodiment of the heart stimulation device, the control circuit is configured to change at least one pacing parameter and determine if this results in a change in the detected regularity and/or in the detected repetitive operation pattern. By changing a pacing parameter and by determining if this results in a corresponding, expected, change in the detected regularity or repetitive operation pattern, it can be determined whether the mentioned PMT is likely to be the case.

The concept “pacing parameter” or the like is meant to encompass any setting that influences the operation of the heart stimulation device. Such settings include different programmed times and different sensitivities for detecting signals.

According to a further embodiment of the heart stimulation device, the pacing parameter that is changed is the time between a sensed event of the first ventricle sensing circuit and the subsequent pacing pulse, during the same time cycle, delivered with the second ventricle pacing circuit. Such a change will result in a change in the operation of the heart stimulation device and the change in the operation can then be used to determine whether the mentioned PMT is the case.

According to a further embodiment of the heart stimulation device, the control circuit is arranged to determine a measure related to the rate, or cycle length, with which the heart stimulation device operates before and after the change of the pacing parameter and to determine the time between a pacing pulse delivered with the second ventricle pacing circuit and the subsequent event sensed by the first ventricle sensing circuit before and after the change of the pacing parameter, and based on this information to determine whether said loop of events would be likely to be the case if the heart stimulation device were in operation in a patient. Such determinations can be used to decide whether the mentioned PMT is likely to be the case. How this can be done will be described in more detail below.

According to a further embodiment of the heart stimulation device, the repetitive operation pattern is V2, R1, V2, R1, V2, R1 . . . , where V2 is a pacing pulse delivered with the second ventricle pacing circuit and R1 is a sensed event of the first ventricle sensing circuit. The sensed event R1 can be an R-wave or a T-wave.

That the repetitive operation pattern is V2, R1, V2, R1, V2, R1 . . . , preferably means that no other events (in an atrium or a ventricle) than the R1 events are sensed, at least not outside the refractory periods of the heart stimulation device, as long as this pattern occurs.

Another possible repetitive operation pattern is V2, P, R1, V2, P, R1, V2, P, R1, . . . .

According to a further embodiment of the heart stimulation device, the operation pattern criterion is that the operation pattern lasts longer than a predetermined time or that the number of paced and sensed events in said operation pattern is higher than a predetermined number. If the operation pattern lasts a long time, then it is likely that the mentioned PMT is the case.

Another aspect of the invention concerns providing a device that is actually able to break (terminate) the detected loop of events. In accordance with this aspect of the invention, the control circuit is arranged to break the operation pattern with which the heart stimulation device operates by changing the operation of the heart stimulation device, wherein the change in operation is done in a manner particularly adapted to be likely to stop the particular, above defined, loop of events that would be likely to be the case if the heart stimulation device were in operation in a patient.

According to a further embodiment of the heart stimulation device, the change in operation involves preventing at least one pacing pulse of the second ventricle pacing circuit from being generated.

According to a further embodiment of the heart stimulation device, the control circuit is configured to prevent the aforementioned at least one pacing pulse of the second ventricle pacing circuit from being generated by one or more of the following measures:

a) simply inhibiting at least one such pacing pulse and thereafter return to the normal operation of the heart stimulation device,

b) changing a refractory period of the operation of the heart stimulation device, such that the heart stimulation device does not react on at least one sensed event of the first ventricle sensing circuit, and thereafter return to the normal operation of the heart stimulation device,

c) changing a blanking period of the operation of the heart stimulation device, such that the heart stimulation device does not sense at least one event that would otherwise be sensed by the first ventricle sensing circuit, and thereafter return to the normal operation of the heart stimulation device,

d) changing a sensitivity setting of the operation of the heart stimulation device, such that the heart stimulation device does not sense at least one event that would otherwise be sensed by the first ventricle sensing circuit, and thereafter return to the normal operation of the heart stimulation device with the normal sensitivity setting,

e) delivering a pacing pulse at least substantially simultaneously with a first ventricular pacing circuit, and the second ventricle pacing circuit.

It will be explained further below how these different manners can be used to break the detected loop of events.

According to a further embodiment of the heart stimulation device, the change in operation involves temporarily shortening the pacing parameter which is the time between an event sensed by the first ventricle sensing circuit and the subsequent pacing pulse, during the same time cycle, delivered with the second ventricle pacing circuit, and thereafter return to the normal operation of the heart stimulation device. By shortening the mentioned pacing parameter, it is less likely that the electrical activity caused by a pacing pulse to the second ventricle will be transferred and sensed in the first ventricle. Consequently, it can be possible to break the operation pattern in this manner.

Another aspect of the invention concerns providing a device that is actually able to prevent future occurrences of the loop of events of the above defined kind. In accordance with this aspect of the invention, the control circuit is also configured to be able to prevent such future occurrences of the loop of events if the heart stimulation device is in operation in a patient, by changing one or more pacing parameters, wherein the change in pacing parameters is done in a manner particularly adapted to prevent the mentioned loop of events.

According to a further embodiment of the heart stimulation device, the device has a memory and the control circuit is configured to store in the memory a sequence of heart stimulation device events, such that, based on an analysis of this sequence of heart stimulation device events, it would be possible to determine the of the said loop of events. By analyzing the sequence of events, it is thus possible to determine the likely cause of the endless loop with which the heart stimulation device operates. The sequence of heart stimulation device events can for example be analysed by a physician during a medical check-up.

It should be noted that the expression “heart stimulation device events” includes both sensed events and delivered pacing pulses.

According to a further embodiment of the heart stimulation device, the control circuit is also configured to carry out an analysis of the stored sequence of heart stimulation device events, and based on this analysis, to automatically change the one or more pacing parameters, such that a future loop of events of the above defined kind is prevented if the heart stimulation device is in operation in a patient. According to this alternative, the device itself thus automatically carries out the necessary change in pacing parameters in order to prevent future occurrences of the endless loop of events.

According to a further embodiment of the heart stimulation device, the control circuit is configured to make the change in pacing parameters in order to prevent a future loop of events of the above defined kind by one or more of the following measures:

a) changing at least one sensitivity setting of the operation of the heart stimulation device, such that the heart stimulation device is less sensitive, and thus less likely to sense at least one kind of event, the sensing of which could trigger the above defined loop of events if the heart stimulation device is in operation in a patient,

b) changing at least one sensitivity setting of the operation of the heart stimulation device, such that the heart stimulation device is more sensitive, and thus more likely to sense at least one kind of event, the lack of sensing of which could trigger the above defined loop of events if the heart stimulation device is in operation in a patient,

c) changing the energy of the pacing pulses delivered by the heart stimulation device,

d) extending at least one blanking or refractory period of the operation of the heart stimulation device, such that the heart stimulation device is less likely to react on at least one kind of event, which, if the heart stimulation device reacted on it, could trigger the above defined loop of events if the heart stimulation device is in operation in a patient.

It will be explained further below how these measures may prevent future occurrences of the loop of events.

According to a further embodiment of the heart stimulation device, the device circuit also has a first atrial sensing and/or pacing circuit, adapted to communicate with a first atrial sensing and/or pacing electrode suited to be positioned in an atrium of a heart, wherein the first atrial sensing and/or pacing circuit is adapted to enable sensing and/or pacing of such an atrium. The present invention is advantageous to use in connection with a heart stimulation device that also has the ability to sense and/or pace at least one atrium.

Another object of the invention is to provide a method of, in a patient who is treated with a bi-ventricular cardiac heart stimulation device, detecting a pacemaker-mediated tachycardia of the above defined kind.

This object of the invention is achieved by a method that includes the following steps:

detect one or both of the following:

i) the regularity of one or more repetitious events related to the ventricles,

ii) a repetitive operation pattern of the heart stimulation device, determine one or both of the following:

iii) if the detected regularity fulfils a predetermined regularity criterion,

iv) if the operation pattern fulfils a predetermined operation pattern criterion, and,

based on the above steps, decide whether the above defined pacemaker-mediated tachycardia is likely to be the case.

The method of the invention also concerns breaking a pacemaker-mediated tachycardia of the above defined kind and preventing future occurrences of such a pacemaker-mediated tachycardia. Different manners of carrying out the method of the invention are described below.

The method of the invention has advantages corresponding to those described above in connection with the device according to the invention.

### BRIEF DESCRIPTION OF THE DRAWINGS

FIG. 1 schematically illustrates a heart stimulation system with a heart stimulation device connected to leads with sensing and pacing electrodes positioned in a heart, in accordance with the present invention.

FIG. 2 schematically illustrates an embodiment of a control circuit in the heart stimulation device of FIG. 1.

FIG. 3 schematically illustrates a time scale for events that may occur in the heart stimulation device of FIG. 1.

FIG. 4 is a flow chart for an embodiment of a method according to the present invention.

### DESCRIPTION OF PREFERRED EMBODIMENTS

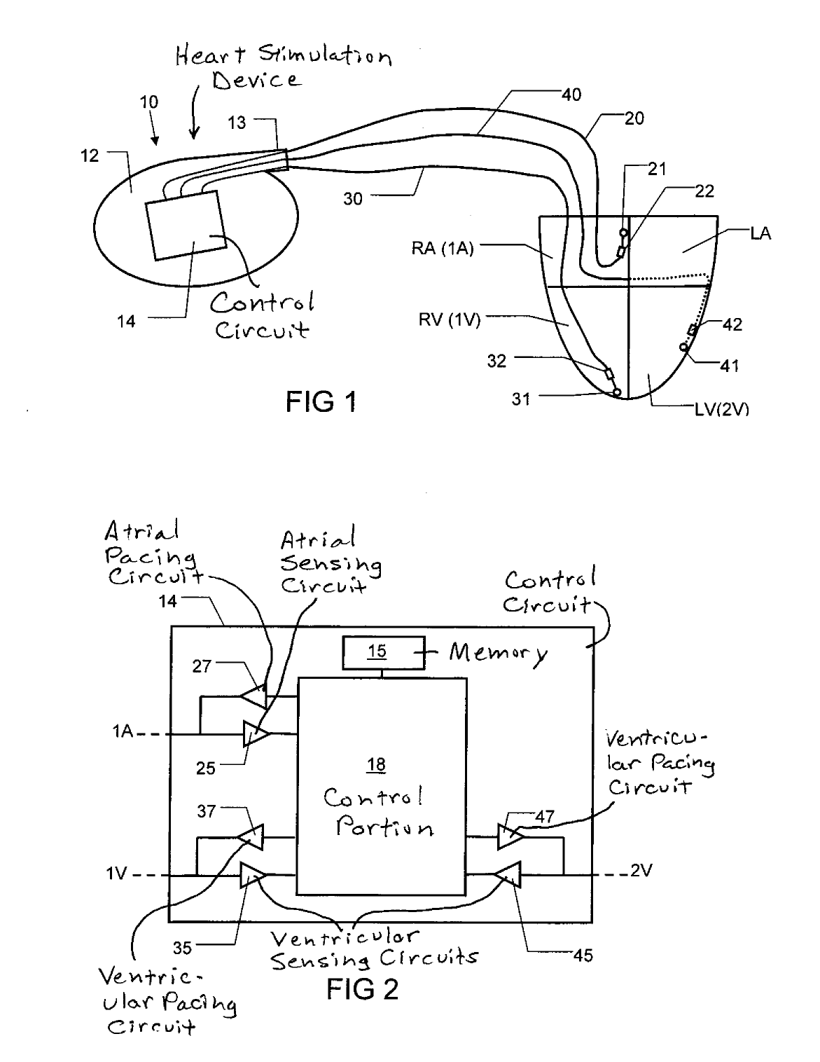

FIG. 1 shows schematically an embodiment of an implantable heart stimulation device 10 according to the invention. The device 10 has a housing 12. The housing 12 includes a control circuit 14. The device 10 has a connector portion 13. Via the connector portion 13, the device 10 can be connected to different leads. In FIG. 1 the device 10 is connected to three leads 20, 30 and 40.

The lead 20 includes a pacing and sensing electrode 21, 22. In the shown example, this electrode 21, 22 is a bipolar electrode with a tip portion 21 and a ring portion 22. However, it is within of the scope of the invention that instead unipolar electrodes can be used, as is known to those skilled in the art. Similarly to the lead 20, the lead 30 includes a pacing and sensing electrode 31, 32 and the lead 40 includes a pacing and sensing electrode 41, 42. The device 10 together with the leads 20, 30, 40 and the electrodes 21, 22; 31, 32; 41 42 can be said to constitute an implantable heart stimulation system.

FIG. 1 also schematically illustrates a heart with a right atrium RA, a left atrium LA, a right ventricle RV and a left ventricle LV.

The electrode 21, 22 constitutes a first atrial sensing and/or pacing electrode 21, 22 which is positioned in a first atrium 1A of the heart, according to this embodiment the right atrium RA, in order to enable sensing and/or pacing of this atrium RA.

The electrode 31, 32 constitutes a first ventricular sensing and pacing electrode 31, 32, which is positioned in a first ventricle 1V of the heart, in this embodiment the right ventricle RV. The first ventricular sensing and pacing electrode 31, 32 is adapted to enable sensing and pacing of this first ventricle 1V.

The electrode 41, 42 constitutes a second ventricular sensing and pacing electrode 41, 42, which is positioned at a second ventricle 2V of the heart, in this embodiment the left ventricle LV. The second ventricular sensing and pacing electrode 41, 42 is adapted to enable sensing and pacing of this second ventricle 2V. The lead 40 may for example be introduced via the right atrium RA and the coronary sinus such that the electrode 41, 42 is positioned in for example the middle or great cardiac vein of the heart. How to introduce the lead 40 in this manner is known to a person skilled in the art.

Although not shown in FIG. 1, it is also possible that the system is connected to further leads and/or further electrodes, for example electrodes positioned in order to sense and/or pace the left atrium LA and electrodes designed to enable defibrillation.

FIG. 2 shows schematically the control circuit 14 in more detail. The control circuit 14 includes a memory 15 connected to a control portion 18. The control circuit 14 includes a first atrial sensing and/or pacing circuit 25, 27. In this embodiment, this circuit 25, 27 includes a sensing circuit 25 and a pacing circuit 27. The first atrial sensing and/or pacing circuit 25, 27 communicates with the first atrial sensing and/or pacing electrode 21, 22 via the lead 20. The first atrial sensing and/or pacing circuit 25, 27 is thus adapted to sense and/or pace an atrium 1A, in this case the right atrium RA.

The control circuit 14 also includes a first ventricular sensing circuit 35 and a first ventricular pacing circuit 37. These circuits 35, 37 communicate with the first ventricular sensing and pacing electrode 31, 32 via the lead 30. The circuits 35, 37 are thus adapted to sense and pace a first ventricle 1V, in this case the right ventricle RV.

The control circuit 14 also includes a second ventricular sensing circuit 45 and a second ventricular pacing circuit 47. These circuits 45, 47 communicate with the second ventricular sensing and pacing electrode 41, 42 via the lead 40. These circuits 45, 47 are adapted to sense and pace a second ventricle 2V, in this case the left ventricle LV.

The control circuit 14 is arranged, or programmed, to include several operational features.

As is normal in a heart stimulation device, the first ventricular sensing circuit 35 and the second ventricular sensing circuit 45 are able to sense events typical for an R-wave (QRS-complex) in the respective ventricle. The ventricular sensing mentioned in this application is thus primarily designed to sense R-waves. However, it should be noted that the ventricular sensing circuits 35, 45 may sometimes also sense a T-wave (ventricular repolarization). Consequently, the operation of the device 10 may depend not only on whether an R-wave is sensed but also on a sensed T-wave.

As is also normal in a heart stimulation device, the first atrial sensing and/or pacing circuit 25, 27 is arranged to be able to detect events typical for a P-wave.

The control circuit 14 is arranged to be able to operate with time cycles corresponding to normal heart cycles. Such an operation is normal for an implantable heart stimulation device. The time cycles are determined by preset timer intervals which also may depend on detected signals.

The control circuit 14 is also arranged to, within a time cycle, be able to deliver pacing pules pulses with both said the first ventricular pacing circuit 37 and said the second ventricular pacing circuit 47 with a time gap VV, during the normal operation of the device 10, between a pacing pulse delivered, or inhibited, by one of said the first ventricular pacing circuit 37 and the second 47 ventricular pacing circuits circuit and a pacing pulse delivered, or inhibited, by the other one of those said first 37 and second 47 ventricular pacing circuits 37 and 47, wherein said the time gap VV is ≧0. A typical value of VV can be between 0 ms and 80 ms, for example 30 ms. For example, if the sense/pace channel 35, 37 is the channel that is normally first with regard to the VV time gap, then, if the first ventricular sensing circuit 35 senses an event, then a pacing pulse can be delivered with the second ventricular pacing circuit 47 after the time VV. The delivery of the pacing pulse with the second ventricular pacing circuit 47 can also be inhibited if the second ventricular sensing circuit 45 senses an event before the pacing pulse is delivered with the second ventricular pacing circuit 47.

It should also be noted that the control circuit 14 can be arranged to operate in the following manner: if an event is sensed by the ventricular sensing circuit (for example the second ventricular sensing circuit 45) that is associated with the ventricle that is normally paced last (i.e. after the time gap VV) during a time cycle when no event has been sensed by the other ventricular sensing circuit (in this example the first ventricular sensing circuit 35) and no pacing pulse has been delivered with the corresponding ventricular pacing circuit (in this example the first ventricular pacing circuit 37), then a pacing pulse is immediately, or at least almost immediately, delivered with this pacing circuit (in this example thus the first ventricular pacing circuit 37). In order to distinguish between the normal time gap W explained above and the short delay “immediately, or at least almost immediately” that has just been explained, herein the label VVᵢ is used for the latter. VVᵢ is ≧0, but normally quite short, for example 10 ms. The time gap that is ≧0 that is mentioned in, inter alia, claim 1 below can thus either be the time gap VV or the time gap VVᵢ.

As is normal for implantable heart stimulation devices, the device 10 is also normally set up to operate with PV and AV delays. PV can for example be defined as the time between the sensing with said first atrial sensing and/or pacing circuit 25, 27 and a subsequent pacing pulse, which may also be inhibited, of the first ventricular pacing circuit 37. AV can for example be defined as the time between the pacing with said first atrial sensing and/or pacing circuit 25, 27 and a subsequent pacing pulse, which may also be inhibited, of the first ventricular pacing circuit 37. It is well known to those skilled in the art how an implantable heart stimulation device is set up in order to operate with PV and AV delays. Furthermore, the device 10 is normally set up to operate with well known blanking and refractory periods.

Although not described in any detail here, the control circuit 14 can be arranged to include several other operational features that are known in connection with heart stimulation devices. Such features include, for example, the ability to detect evoked responses to delivered pacing pulses (such detection is normally done with a detection logic that is different from that used for detecting R-waves or T-waves); the ability to deliver back-up pulses if a heart chamber is not captured when a pacing pulse has been delivered; the ability to perform capture threshold searches; the ability to sense the physiological activity of the patient in whom the device has been implanted; the ability to carry out defibrillation; the ability to communicate with the help of so-called telemetry, etc.

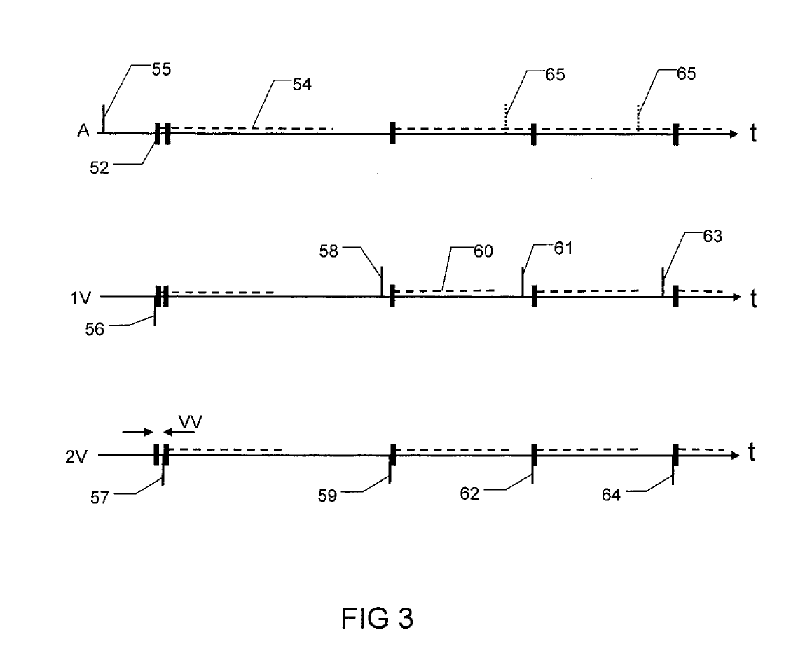

FIG. 3 shows schematically on a common time scale heart stimulation device events related to an atrial channel A, a first ventricular channel 1V and a second ventricular channel 2V. The black markers (like the mark 52) indicate a blanking period for the respective channel. During the blanking period, the sense circuit in question is disabled and no sensing is thus possible during the blanking periods. The hatched lines (like the line 54) indicate refractory periods for the respective channels. The heart stimulation device will not react on possible events that are sensed during such refractory periods. A line pointing upwardly (for example the lines 55 and 58) indicates a sensed event in the respective channel and a line pointing downwardly (like the lines 56 and 57) indicates a pacing pulse delivered by the respective channel.

FIG. 3 illustrates a chain of events that may occur in a bi-ventricular heart stimulation device. 55 indicates an event sensed by the first atrial sensing circuit 25. After a certain delay (AV-delay) a pacing pulse 56 is delivered with the first ventricular pacing circuit 37. After the time Vv a pacing pulse 57 is delivered with the second ventricular pacing circuit 47. A certain sensed event (or the lack of sensing of a certain event that should have been sensed) may cause the heart stimulation device 10 to operate in an endless loop. FIG. 3 illustrates that the first ventricular sensing circuit 35 senses an event 58. This sensing can for example constitute oversensing, i.e. the sensing is not caused by any real cardiac event that should have been sensed. This sensed event 58 causes a pacing pulse 59 to be delivered with the second ventricular pacing circuit 47 after a time gap VV. The pacing pulse 59 causes depolarization of the second ventricle 2V with an associated electrical activity. This electrical activity may also involve a repolarization (T-wave). The electrical activity is transferred via the heart tissue to the first ventricle 1V. This transfer takes a certain time. Since the transfer takes a certain time it can “arrive” in the first ventricle 1V after the refractory period 60. This transferred electrical activity is sensed as an event 61 by the first ventricular sensing circuit 35. The event 61 causes the generation of a pacing pulse 62 with the second ventricular pacing circuit 47 after the time gap VV. Because of the mentioned transfer time and the W time gap, the pacing pulse 62 is emitted to the second ventricle 2V after the biological refractory period caused by the previous pacing pulse 59. The second ventricle 2V is therefore captured again by the pacing pulse 62. Consequently, the second ventricle 2V is depolarized and an associated electrical activity, which may also involve a repolarization, is transferred via the heart tissue to the first ventricle 1V and is there sensed again as an event 63. The sensed event 63 causes another pacing pulse 64 to be generated by the second ventricular pacing circuit 47. An endless loop of events is thus the case, i.e. a PMT has been generated.

It should be noted that the events 65 and 66 in FIG. 3 illustrate retrograde P-waves occurring in the atrial channel. However, these events 65, 66 do not cause any change in the operation of the heart stimulation device 10, since these events 65, 66 fall within the refractory period for the atrial channel (post ventricular atrial refractory period, PVARP).

It should also be noted that although not shown in FIG. 3, it is possible that the first ventricle 1V is in fact the ventricle that is normally associated with the ventricular channel that is the last channel with regard to the VV time gap. A sensing in such a channel can cause an almost immediate (after the time gap VVᵢ) generation of a pacing pulse with the other ventricular channel. This pacing pulse may cause a depolarization with an associated electrical activity in the ventricle in question. This electrical activity is transferred via the heart tissue to the other ventricle and a similar endless loop of events to that described in connection with FIG. 3 may occur.

According to the present invention, the control circuit 14 is arranged to be able to detect whether the above described loop of events is likely to be the case by performing the following steps:

detect one or both of the following:

i) the regularity of one or more repetitious events related to the first ventricular sensing circuit 35 or the second ventricular pacing circuit 47,

ii) a repetitive operation pattern of the heart stimulation device 10, determine one or both of the following:

iii) if the detected regularity fulfils a predetermined regularity criterion,

iv) if the operation pattern fulfils a predetermined operation pattern criterion, and,

based on the above steps, determine whether the loop of events would be likely to be the case if the heart stimulation device 10 were in operation in a patient.

The mentioned regularity can be one or more of the following repetitious events:

a) the time between consecutive sensed events of said first ventricular sensing circuit 35,

b) the time between consecutive pacing pulses delivered with said second ventricular pacing circuit 47,

c) the time between a pacing pulse delivered with said second ventricular pacing circuit 47 and the subsequent sensed event of said first ventricular sensing circuit 35.

The regularity criterion can be that a measure of how much the respective time varies is below a predetermined value. If a PMT of the described kind is the case, the time between the respective repetitious event will be very stable. The regularity criterion can thus be that the standard deviation or the coefficient of variance (standard deviation divided by the average) is small for the respective time. In order to determine the standard deviation or the coefficient of variance, the times between the respective consecutive events should be monitored over several time cycles, for example over at least 8 time cycles. It is not necessary to determine the regularity of all the mentioned repetitious events a), b) and c). However if more than one of these repetitious events are monitored, then the accuracy in the determination of whether a PMT of the above-mentioned kind is the case will increase.

The mentioned repetitive operation pattern to be detected can be the repetitive operation pattern V2, R1, V2, R1, V2, R1 . . . , where V2 is a pacing pulse delivered with the second ventricular pacing circuit 47 and R1 is a sensed event of the first ventricular sensing circuit 35. The operation pattern criterion can thereby be that the operation pattern lasts longer than a predetermined time, for example longer than 8 s, or that the number of paced and sensed events in said operation pattern is higher than a predetermined number, for example higher than 10.

In order to further improve the detection of whether the above described loop of events is likely to be the case, the control circuit 14 can be arranged to change at least one pacing parameter and determine if this results in a change in the detected regularity and/or in the detected repetitive operation pattern. The pacing parameter that is changed can be the time VV or VVᵢ between a sensed event of the first ventricular sensing circuit 35 and the subsequent pacing pulse, during the same time cycle, delivered with the second ventricular pacing circuit 47. The control circuit 14 can be arranged to determine a measure related to the rate, or cycle length, with which the heart stimulation device 10 operates before and after the change of the pacing parameter and to determine the time between a pacing pulse delivered with the second ventricular pacing circuit 47 and the subsequent event sensed by the first ventricular sensing circuit 35 before and after the change of the pacing parameter, and based on this information to determine whether said loop of events would be likely to be the case if the heart stimulation device 10 were in operation in a patient. If for example the time W is increased with 20 ms, then, if the mentioned PMT is the case, also the time between consecutive sensed events (like 61 and 63), or the time between consecutive paced events (like 62 and 64) will increase with 20 ms. However, the time between a paced event (like 62) and the following sensed event (like the event 63) will not change.

It should be noted that in order to increase the accuracy of the detection of the PMT, the different described manners of detecting such a PMT may be combined in one and the same heart stimulation device 10.

Once the above described loop of events has been detected, the control circuit 14 can also be arranged to break, or terminate, the operation pattern with which the heart stimulation device 10 operates by changing the operation of the heart stimulation device 10. The change in operation is done in a manner particularly adapted to be likely to stop the particular, above defined, loop of events that would be likely to be the case if the heart stimulation device 10 were in operation in a patient. The change in operation can involve preventing at least one pacing pulse of said second ventricular pacing circuit 47 from being generated. This can be done by one or more of the following measures:

a) inhibiting at least one such pacing pulse and thereafter return to the normal operation of the heart stimulation device 10,

b) changing a refractory period of the operation of the heart stimulation device 10, such that the heart stimulation device 10 does not react on at least one sensed event of the first ventricular sensing circuit 35, and thereafter return to the normal operation of the heart stimulation device 10,

c) changing a blanking period of the operation of the heart stimulation device 10, such that the heart stimulation device 10 does not sense at least one event that would otherwise be sensed by the first ventricular sensing circuit 35, and thereafter return to the normal operation of the heart stimulation device 10,

d) changing a sensitivity setting of the operation of the heart stimulation device 10, such that the heart stimulation device 10 does not sense at least one event that would otherwise be sensed by the first ventricular sensing circuit 35, and thereafter return to the normal operation of the heart stimulation device 10 with the normal sensitivity setting,

e) delivering a pacing pulse at least substantially simultaneously with the first ventricular pacing circuit 37, and the second ventricular pacing circuit 47.

The different points a) to e) will now be explained.

a): If a pacing pulse is not delivered by the second ventricular pacing circuit 47, then no depolarization will occur in the second ventricle, which means that no electrical activity will be transferred to the first ventricle 1V. Consequently, the PMT is broken.

b): The ventricular refractory period 60 of the first ventricular sensing circuit 35 may be increased such that the heart stimulation device 10 will not react on the sensed event (like 61) transferred from the second ventricle 2V.

c): Similarly, if a blanking period is extended for such a long time that a transferred event is not sensed, then the PMT will be broken.

d): If the first ventricular sensing circuit 35 is made less sensitive, a transferred electrical activity (like the event 61) will not be sensed by the first ventricular sensing circuit 35. Consequently, the PMT will be broken.

e): If pacing pulses are delivered simultaneously by the first ventricular pacing circuit 37 and the second ventricular pacing circuit 47, both the ventricles 2V, 1V will be biologically refractory after the delivery of such pacing pulses. Consequently, it is less likely that a transferred electrical activity will depolarize the first ventricle 1V. If the first ventricle is not depolarized, no event (like the event 61) will occur and be sensed by the first ventricular sensing circuit 35. This will lead to the fact that no pacing pulse (like the pacing pulse 62) will be delivered by the second ventricular pacing circuit 47. The pacing pulse 62 will thus be inhibited, and the PMT will be broken.

Another possible manner of changing the operation of the device 10 in order try to break the mentioned loop of events involves arranging the control circuit 14 to temporarily shorten the pacing parameter VV (or VVᵢ) which is the time between an event sensed by the first ventricular sensing circuit 35 and the subsequent pacing pulse, during the same time cycle, delivered with the second ventricular pacing circuit 47, and thereafter return to the normal operation of the heart stimulation device 10. If VV is made shorter, then it is more likely that the first ventricle 1V is biologically refractory when the electrical activity is transferred from the second ventricle 2V. Consequently, it is less likely that such a transferred electrical activity will depolarize the first ventricle 1V. Therefore, the PMT can be broken.

The control circuit 14 can also be configured to be able to prevent, or aid in preventing, future occurrences of the loop of events of the above defined kind if the heart stimulation device 10 is in operation in a patient. This can be done by changing one or more pacing parameters, wherein the change in pacing parameters is done in a manner particularly adapted to pre-vent the mentioned loop of events. The control circuit 14 can hereby be arranged to store in the memory 15 a sequence of heart stimulation device events, such that, based on an analysis of this sequence of heart stimulation device events, it would be possible to determine the cause said loop of events. This analysis can be performed by a physician at a medical check-up, when, for example, via telemetry the physician is informed of the sequence of heart stimulation device events that have been stored in the memory 15. Based on this analysis, the physician can reprogram the device 10 such that future occurrences of the loop of events will be prevented. Alternatively, the control circuit 14 can be arranged to automatically carry out an analysis of the stored sequence of heart stimulation device events, and based on this analysis, to automatically change one or more pacing parameters, such that a future loop of events of the above defined kind is prevented if the heart stimulation device 10 is in operation in a patient.

The control circuit 14 can be configured such that the change in pacing parameters in order to prevent a future loop of events of the above defined kind involves one or more of the following measures:

a) changing at least one sensitivity setting of the operation of the heart stimulation device 10, such that the heart stimulation device 10 is less sensitive, and thus less likely to sense at least one kind of event, the sensing of which could trigger the above defined loop of events if the heart stimulation device 10 is in operation in a patient,

b) changing at least one sensitivity setting of the operation of the heart stimulation device 10, such that the heart stimulation device 10 is more sensitive, and thus more likely to sense at least one kind of event, the lack of sensing of which could trigger the above defined loop of events if the heart stimulation device 10 is in operation in a patient,

c) changing the energy of the pacing pulses delivered by the heart stimulation device 10,

d) extending at least one blanking or refractory period of the operation of the heart stimulation device 10, such that the heart stimulation device 10 is less likely to react on at least one kind of event, which, if the heart stimulation device 10 reacted on it, could trigger the above defined loop of events if the heart stimulation device 10 is in operation in a patient.

Below follow some explanations concerning these measures a) to d).

a): The PMT can be initiated by oversensing. By making the heart stimulation device 10 (or at least the relevant atrial or ventricular channel of the device 10) less sensitive, it is less likely that oversensing will occur. Consequently, it is less likely that a PMT will be initiated.

b): A PMT can also be initiated by undersensing. If this is the case, then future initiation of the PMT can be prevented by making the heart stimulation device 10 (or the relevant channel of the device 10) more sensitive.

c): A PMT can also be caused by a loss of capture, i.e. by the fact that a certain heart chamber is not captured by a generated pacing pulse. If the energy of the pacing pulses is increased, the probability for a loss of capture decreases. It is, for example, possible for the control circuit 14 to perform a capture threshold test in order to determine an appropriate energy of the pacing pulses such that loss of capture is avoided.

d): By extending blanking or refractory periods, it can be avoided that transferred electrical activity, of the kind explained above, will be sensed by the first ventricular sensing circuit 35. Consequently, it can be avoided that a PMT is initiated.

The invention also concerns a method of, in a patient who is treated with a bi-ventricular cardiac heart stimulation device, detecting a pacemaker-mediated tachycardia of the above defined kind.

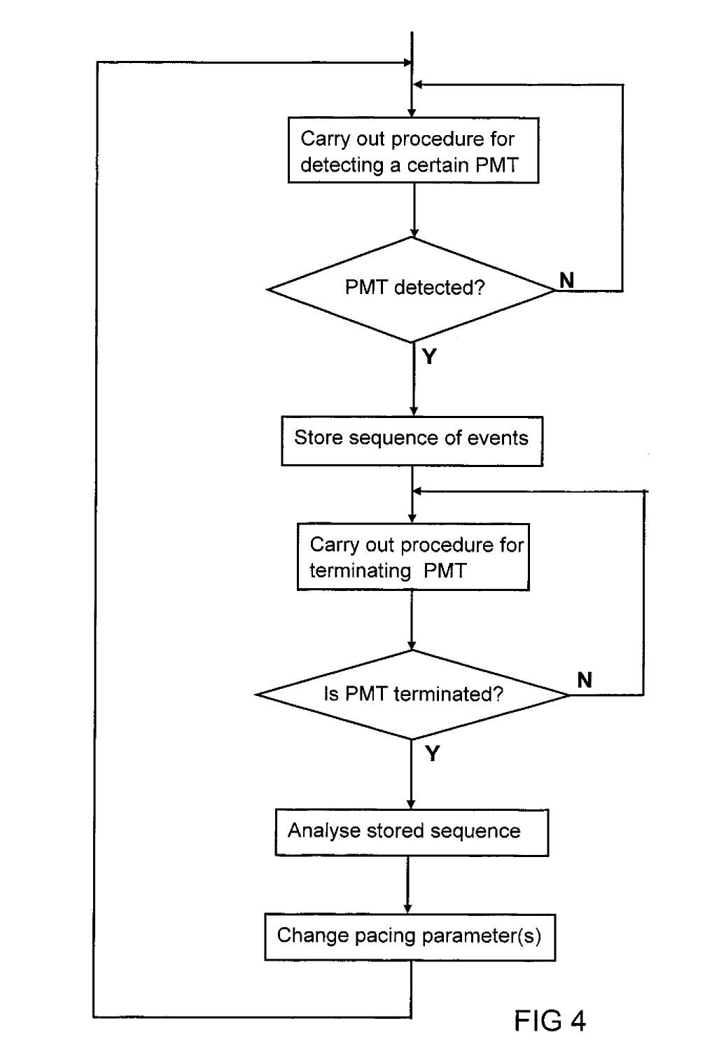

A schematic flow chart illustrating a method according to the invention is shown in FIG. 4. According to the method, a procedure for detecting a PMT of the above described particular kind is carried out. If a PMT is detected, then the sequence of events that characterises characterizes the PMT is stored in a memory. Furthermore, the PMT is terminated. The sequence of events stored in the memory can then be analysed. This can be done either automatically by the device 10 itself or by a physician at a medical check-up. Thereafter, pacing parameters concerning the operation of the heart stimulation device 10 are changed in order to prevent future occurrences of the PMT. The change of pacing parameters can either be performed by a physician or automatically by the device 10 itself.

The method according to the invention will now be described in some more detail. However, since the method in may respects corresponds to the operation of the heart stimulation device 10 explained above, specific details of the method which correspond to the operation of the device are omitted below. The method according to the invention thus includes the following steps:

detect one or both of the following:

i) the regularity of one or more repetitious events related to the ventricles,

ii) a repetitive operation pattern of the heart stimulation device, determine one or both of the following:

iii) if the detected regularity fulfils a predetermined regularity criterion,

iv) if the operation pattern fulfils a predetermined operation pattern criterion, and,

based on the above steps, decide whether the above defined pacemaker-mediated tachycardia is likely to be the case.

It should be mentioned that, preferably, the method also includes the step of providing means for sensing and/or pacing in at least a first atrium of the heart.

The detection of said regularity can involve detecting the regularity of one or more of the following repetitious events:

a) the time between consecutive sensed events of the first ventricle,

b) the time between consecutive pacing pulses delivered to the second ventricle,

c) the time between a pacing pulse to the second ventricle and the subsequent sensed event of the first ventricle.

The regularity criterion can be that a measure of how much the respective time varies is below a predetermined value.

Analogously to the above description of the device 10 according to the invention, the repetitive operation pattern can be V2, R1, V2, R1, V2, R1 . . . , where V2 is a pacing pulse to the second ventricle and R1 is a sensed event of said first ventricle. The operation pattern criterion can be that the operation pattern lasts longer than a predetermined time or that the number of paced and sensed events in said operation pattern is higher than a predetermined number.

Furthermore, the method may include the step of changing at least one pacing parameter and determine if this results in a change in the detected regularity and/or in the detected repetitive operation pattern. The pacing parameter that is changed can be the time VV or VVᵢ between a sensed event in the first ventricle and the subsequent pacing pulse, during the same time cycle, to the second ventricle. The method can thereby include the step of determining a measure related to the rate, or cycle length, with which the heart stimulation device operates before and after the change of the pacing parameter and also determining the time between a pacing pulse to the second ventricle and the subsequent sensed event in the first ventricle before and after the change of the pacing parameter. Based on this information, it can be determined whether the above defined pacemaker-mediated tachycardia is (likely to be) the case.

The method can also include steps for breaking, or terminating, the detected, above defined, pacemaker-mediated tachycardia. This can be done by changing the operation of the heart stimulation device, wherein the change in operation is done in a manner particularly adapted to stop the particular, above defined, pacemaker-mediated tachycardia that has been detected. This change in operation can involve preventing at least one pacing pulse to the second ventricle from being generated. This can be done by one or more of the following measures:

a) simply inhibiting at least one such pacing pulse and thereafter return to the normal operation of the heart stimulation device,

b) changing a refractory period of the operation of the heart stimulation device, such that the heart stimulation device does not react on at least one sensed event in the first ventricle, and thereafter return to the normal operation of the heart stimulation device,

c) changing a blanking period of the operation of the heart stimulation device, such that the heart stimulation device does not sense at least one event in the first ventricle, and thereafter return to the normal operation of the heart stimulation device,

d) changing a sensitivity setting of the operation of the heart stimulation device, such that the heart stimulation device does not sense at least one event in the first ventricle, and thereafter return to the normal operation of the heart stimulation device with the normal sensitivity setting,

e) deliver a pacing pulse at least substantially simultaneously to the first and the second ventricle.

Another manner of breaking the loop of events can be to temporarily shorten the pacing parameter VV (or VVᵢ) which is the time between a sensed event in the first ventricle and the subsequent pacing pulse, during the same time cycle, to the second ventricle, and thereafter return to the normal operation of the heart stimulation device.

The method of the invention can also include preventing future pacemaker-mediated tachycardias, of the above defined kind. This can be done by changing one or more pacing parameters, wherein the change in pacing parameters is done in a manner particularly adapted to prevent the particular pacemaker-mediated tachycardia that has been detected. This method can include recording and analyzing a sequence of heart stimulation device events, and, based on this analysis, determine the cause of the pacemaker-mediated tachycardia, and change said one or more pacing parameters, based on the analysis, such that future pacemaker-mediated tachycardia of the above defined kind is prevented. The change in pacing parameters in order to prevent future pacemaker-mediated tachycardia of the above defined kind, can involve one or more of the following measures:

a) changing at least one sensitivity setting of the operation of the heart stimulation device, such that the heart stimulation device is less sensitive, and thus less likely to sense at least one kind of event in the heart, the sensing of which could trigger the above defined pacemaker-mediated tachycardia,

b) changing at least one sensitivity setting of the operation of the heart stimulation device, such that the heart stimulation device is more sensitive, and thus more likely to sense at least one kind of event in the heart, the lack of sensing of which could trigger the above defined pacemaker-mediated tachycardia,

c) changing the energy of the pacing pulses delivered by the heart stimulation device,

d) extending at least one blanking or refractory period of the operation of the heart stimulation device, such that the heart stimulation device is less likely to react on at least one kind of event in the heart, which, if the heart stimulation device reacted on it, could trigger the above defined pacemaker-mediated tachycardia.

The method according to the invention can, for example, be performed on a human or animal being suffering from congestive heart failure, for example on a on a human or animal being suffering from a bundle branch block.

Although modifications and changes may be suggested by those skilled in the art, it is the intention of the inventor to embody within the patent warranted hereon all changes and modifications as reasonably and properly come within the scope of his contribution to the art.

I claim as my invention:

1-33. (canceled)

34. A heart stimulation device for stimulating a heart having a first ventricle and a second ventricle, said heart stimulation device comprising:
a first ventricle sensing circuit that communicates with a first ventricle sensing electrode configured to be positioned in or at the first ventricle to enable the first ventricle sensing circuit to sense events in the first ventricle;
a second ventricle pacing circuit that communicates with a second ventricle pacing electrode configured to be positioned in or at the second ventricle to enable the second ventricle pacing circuit to deliver pacing pulses to the second ventricle;
a control circuit connected to said first ventricle sensing circuit and to said second ventricle pacing circuit and configured to operate said first ventricle sensing circuit and said second ventricle pacing circuit with time cycles corresponding to normal heart cycles, said control circuit being configured to, within one of said time cycles, since an event in the first ventricle via the first ventricle sensing circuit and, after a time duration that is greater than or equal to zero, cause the second ventricle pacing circuit to deliver a pacing pulse to the second ventricle;
said control circuit being configured to detect a loop of events representative of pacemaker-mediated tachycardia caused by a sensed event in the first ventricle causing delivery of a pacing pulse to the second ventricle, with the pacing pulse delivered to the second ventricle causing a depolarization of the second ventricle with associated electrical activity that is transferred via heart tissue to the first ventricle and which is sensed as an event in the first ventricle that causes a further pacing pulse to be delivered to the second ventricle with a repetition of said loop of events;
said control circuit being configured to identify a presence of said loop of events by detecting at least one of (i) a regularity of one or more repetitious events related to operation of said firsts ventricle sensing circuit or said second ventricle pacing circuit, and (ii) a repetitive operation pattern of the heart stimulation device;
said control circuit being configured to automatically determine at least one of whether the detected regularities satisfies a predetermined regularity criterion, and whether the operation pattern satisfies a predetermined operation pattern criterion; and
said control circuit being configured, depending on whether at least one of said criterion is satisfied, to identify satisfaction of said at least one of said criterion as indicative of a presence of pacemaker-mediated tachycardia.

35. A heart stimulation device as claimed in claim 34 wherein said control circuit is configured to detect said regularity by detecting the regularity of at least one repetitious time selected from the group consisting of a time between consecutive sensed events by said first ventricle sensing circuit, a time between consecutive pacing pulses delivered by said second ventricle pacing circuit, and a time between a pacing pulse delivered by said second ventricle pacing circuit and a subsequent sensed event by said first ventricle sensing circuit.

36. A heart stimulation device as claimed in claim 35 wherein said control circuit is configured to employ, as said regularity criterion, a measure of how much said at least one repetitious time is below a predetermined value.

37. A heart stimulation device as claimed in claim 34 wherein said second ventricle pacing circuit operates with a plurality of pacing parameters, and wherein said control circuit is configured to change at least one of said pacing parameters and to determine if changing said at least one of said pacing parameters causes a change in at least one of the detected regularity or the detected repetitive operation pattern.

38. A heart stimulation device as claimed in claim 37 wherein said control circuit is configured to change said at least one pacing parameter by changing a time between a sensed event by said first ventricle sensing circuit and delivery of a subsequent pacing pulse, during the same time cycle, by said second ventricle pacing circuit.

39. A heart stimulation device as claimed in claim 38 wherein said control circuit is configured to measure a cycle duration of respective time cycles occurring before and after changing said at least one pacing parameter by determining, in the respective cycles, the time between a pacing pulse delivered by said second ventricle pacing circuit and a subsequent event sensed by said first ventricle sensing circuit.

40. A heart stimulation device as claimed in claim 34 wherein said control circuit is configured to use a pattern, as said repetitive operation pattern, of V2, R1, V2, R1, V2, R1 . . . , wherein V2 represents a pacing pulse delivered by said second ventricle pacing circuit and R1 represents a sensed event by said first ventricle sensing circuit.

41. A heart stimulation device as claimed in claim 40 wherein said control circuit is configured to employ, as said operation pattern criterion, a criterion selected from the group consisting of whether said repetitive operation pattern has a duration longer than a predetermined duration, and whether said repetitive operation pattern comprises a number of paced and sensed events that is higher than a predetermined number.

42. A heart stimulation device as claimed in claim 34 wherein said control circuit is configured, upon said determination of the presence of pacemaker-mediated tachycardia, to break the repetitive operation pattern by changing said repetitive operation pattern in a manner likely to stop said loop of events.

43. A heart stimulation device as claimed in claim 42 wherein said control circuit is configured to break said repetitive operation pattern by preventing delivery of at least one pacing pulse by said second ventricle pacing circuit.

44. A heart stimulation device as claimed in claim 43 comprising a first ventricle pacing circuit, and wherein said control circuit is configured to prevent said at least one pacing pulse from being delivered by said second ventricular pacing circuit by a measure selected from the group consisting of inhibiting generation of said at least one pacing pulse by said second ventricle pacing circuit and thereafter returning to said repetitive operation pattern, changing a refractory period in the repetitive operation pattern by inhibiting a reaction to at least one sensed event by said first ventricle sensing circuit and thereafter returning to said repetitive operation pattern, changing a blanking period of the repetitive operation pattern by preventing said first ventricle sensing circuit from sensing at least one event that would otherwise be sensed by said first ventricle sensing circuit and thereafter returning to said repetitive operation pattern, changing a normal sensitivity setting in the repetitive operation pattern to cause said first ventricle sensing circuit not to sense at least one event that would otherwise be sensed by said first ventricle circuit and thereafter returning to said normal sensitivity setting, and causing said first ventricle pacing circuit to deliver a pacing pulse substantially simultaneously with said second ventricle pacing circuit.

45. A heart stimulation device as claimed in claim 42 wherein said control circuit is configured to change said repetitive operation pattern by temporarily shortening a time between an event sensed by said first ventricle sensing circuit and subsequent delivery of a pacing pulse, during the same cycle, by said second ventricle pacing circuit, and thereafter returning to said repetitive operation pattern.

46. A heart stimulation device as claimed in claim 34 wherein said control circuit is configured to, upon determining the presence of pacemaker-mediated tachycardia, prevent a future occurrence of pacemaker-mediated tachycardia by permanently changing said repetitive operation pattern in a manner that prevents a re-occurrence of said loop of events.

47. A heart stimulation device as claimed in claim 46 comprising a memory in which a sequence of events occurring in said heart stimulation device is stored, and wherein said control circuit automatically analyzes said sequence stored in said memory to identify a permanent change to said repetitive operation pattern that is likely to preclude said reoccurrence of said loop of events.

48. A heart stimulation device as claimed in claim 47 wherein said control circuit is configured to permanently change said repetitive operation pattern by a measure selected from the group consisting of changing at least one sensitivity setting to a less sensitive sensitivity setting for an event that is capable of triggering said loop of events, changing at least one sensitivity setting to a more sensitive setting for sensing an event that is likely to trigger said loop of events, changing an energy of the pacing pulses delivered by at least by said second ventricle pacing circuit, and extending at least one of a blanking period or a refractory period of said repetitive operation pattern to make said heart stimulation device less likely to react to an event that is capable of triggering said loop of events.

49. A heart stimulation device as claimed in claim 34 comprising an atrial sensing circuit, an atrial pacing circuit, and at least one atrial electrode configured to be positioned in an atrium of the heart and connected to said atrial sensing circuit and said atrial pacing circuit to enable said atrial sensing circuit to sense events in the atrium and to enable said atrial pacing circuit to deliver pacing pulses to the atrium.

50. A method for, in a patient being paced with a bi-ventricular cardiac stimulation device, detecting pacemaker-mediated tachycardia caused by a sensed event in a first ventricle causing generation of a pacing pulse to a second ventricle that causes depolarization of the second ventricle with an associated electrical activity that is transferred via heart tissue to the first ventricle and that is sensed in the first ventricle, which results in a new pacing pulse being delivered to the second ventricle, forming a loop, said method comprising the steps of:
automatically detecting at least one of (i) a regularity of one or more repetitious events related to said first ventricle and said second ventricle, and (ii) a repetitive operation pattern of the heart stimulation device;
automatically determining if the detected regularity satisfies a predetermined regularity criterion and if the detected operation pattern satisfies a predetermined operation pattern criterion; and
based on whether at least one of said criteria is satisfied, identifying a presence of pacemaker-mediated tachycardia.

51. A method stimulation device as claimed in claim 50 comprising detecting said regularity by detecting the regularity of at least one repetitious time selected from the group consisting of a time between consecutive sensed events in said first ventricle, a time between consecutive pacing pulses delivered to said second ventricle, and a time between a pacing pulse delivered to said second ventricle and a subsequent sensed event in said first ventricle.

52. A method as claimed in claim 51 comprising employing, as said regularity criterion, a measure of how much said at least one repetitious time is below a predetermined value.

53. A method as claimed in claim 50 comprising pacing said second ventricle using a plurality of pacing parameters, and changing at least one of said pacing parameters and determining if changing said at least one of said pacing parameters causes a change in at least one of the detected regularity or the detected repetitive operation pattern.

54. A method as claimed in claim 53 comprising changing said at least one pacing parameter by changing a time between a sensed event in said first ventricle and delivery of a subsequent pacing pulse, during the same time cycle, to said second ventricle.

55. A method as claimed in claim 54 comprising measuring a cycle duration of respective time cycles occurring before and after changing said at least one pacing parameter by determining, in the respective cycles, the time between a pacing pulse delivered to said second ventricle and a subsequent event sensed in said first ventricle.

56. A method as claimed in claim 54 comprising using a pattern, as said repetitive operation pattern, of V2, R1, V2, R1, V2, R1 . . . , wherein V2 represents a pacing pulse delivered by said second ventricle pacing circuit and R1 represents a sensed event by said first ventricle sensing circuit.

57. A method as claimed in claim 56 employing, as said operation pattern criterion, a criterion selected from the group consisting of whether said repetitive operation pattern has a duration longer than a predetermined duration, and whether said repetitive operation pattern comprises a number of paced and sensed events that is higher than a predetermined number.

58. A method as claimed in claim 50 comprising, upon said determination of the presence of pacemaker-mediated tachycardia, automatically breaking the repetitive operation pattern by changing said repetitive operation pattern in a manner likely to stop said loop of events.

59. A method as claimed in claim 58 comprising breaking said repetitive operation pattern by preventing delivery of at least one pacing pulse to said second ventricle.

60. A method as claimed in claim 59 comprising pacing a first ventricle, and preventing said at least one pacing pulse from being delivered to said second ventricular pacing circuit by a measure selected from the group consisting of inhibiting generation of said at least one pacing pulse by a second ventricle pacing circuit and thereafter returning to said repetitive operation pattern, changing a refractory period in the repetitive operation pattern by inhibiting a reaction to at least one sensed event in said first ventricle and thereafter returning to said repetitive operation pattern, changing a blanking period of the repetitive operation pattern by preventing sensing of at least one event that would otherwise be sensed in said first ventricle and thereafter returning to said repetitive operation pattern, changing a normal sensitivity setting in the repetitive operation pattern to cause at least one event that would otherwise be sensed in said first ventricle not to be sensed and thereafter returning to said normal sensitivity setting, and delivering a pacing pulse to said first ventricle substantially simultaneously with delivery of a pacing pulse to said second ventricle.

61. A method as claimed in claim 58 comprising changing said repetitive operation pattern by temporarily shortening a time between an event sensed in said first ventricle sensing circuit and subsequent delivery of a pacing pulse, during the same cycle, to said second ventricle, and thereafter returning to said repetitive operation pattern.

62. A method as claimed in claim 50 comprising, upon determining the presence of pacemaker-mediated tachycardia, automatically preventing a future occurrence of pacemaker-mediated tachycardia by permanently changing said repetitive operation pattern in a manner that prevents a re-occurrence of said loop of events.

63. A method as claimed in claim 62 comprising storing a sequence of events occurring in said heart stimulation device is stored, and automatically analyzing said stored sequence to identify a permanent change to said repetitive operation pattern that is likely to preclude said reoccurrence of said loop of events.

64. A method as claimed in claim 63 comprising permanently changing said repetitive operation pattern by a measure selected from the group consisting of changing at least one sensitivity setting to a less sensitive sensitivity setting for an event that is capable of triggering said loop of events, changing at least one sensitivity setting to a more sensitive setting for sensing an event that is likely to trigger said loop of events, changing an energy of pacing pulses delivered at least to said second ventricle, and extending at least one of a blanking period or a refractory period of said repetitive operation pattern to make said heart stimulation device less likely to react to an event that is capable of triggering said loop of events.

65. A method as claimed in claim 50 sensing events in an atrium of the heart delivering pacing pulses to said atrium.
